(19) United States
(12) Patent Application Publication
(10) Pub. No.: US 2010/0160933 A1
(43) Pub. Date: Jun. 24, 2010

(54) METHODS OF SURGICALLY MODIFYING THE DUODENUM

(75) Inventors: Katie L. Krueger, Merrimack, NH (US); Harold M. Martins, Newton, MA (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 12/646,772

(22) Filed: Dec. 23, 2009

Related U.S. Application Data
(60) Provisional application No. 61/140,797, filed on Dec. 24, 2008.

(51) Int. Cl. A61B 17/128; A61B 17/00

(52) U.S. Cl. 606/139; 128/898

(57) ABSTRACT

The present application provides methods and devices for inducing weight loss. In particular, the present application provides methods for modifying a duodenum to induce weight loss.

### CROSS-REFERENCE TO RELATED APPLICATION

This application claims the benefit of U.S. Provisional Application No. 61/140,797, filed Dec. 24, 2008, the entire contents of which are incorporated herein by reference.

### FIELD OF THE INVENTION

This application pertains to methods for inducing weight loss. More particularly, this application pertains to methods for surgically modifying the duodenum to induce weight loss and thereby treat obesity.

### BACKGROUND OF THE INVENTION

Obesity is the second leading cause of preventable death in the United States, accounting for an estimated 300,000 deaths each year. The spectrum of comorbid conditions associated with obesity includes cancer, type II diabetes, osteoarthritis, hypertension, and heart disease. The economic cost of obesity in the year 2000 was estimated at $117 billion. Treatments include diet and behavioral therapies, pharmacotherapy, and surgery. Non-surgical treatments are utilized for patients with a body-mass index (BMI)>30 and have not proven very effective. Surgical interventions are typically performed only on those patients with a BMI>40 (deemed morbidly obese). Surgical interventions include restrictive operations that reduce the size of the stomach pouch to limit food intake, malabsorptive procedures that rearrange the small intestine in an attempt to decrease the functional length or efficiency of nutrient absorption, or combination procedures which involve both restrictive and malabsorptive operations. One combination procedure dubbed Gastric Bypass (GPB—a.k.a. Roux-en-Y) has been proven effective for most patients, who maintain about 70% of excess weight loss after five years, and 50% at ten years. Gastric banding, a restrictive procedure that reduces the size of the stomach also is somewhat effective. Both of these types of procedures can now be performed laparoscopically, but are not without complications. Moreover, GPB is irreversible. Accordingly, there is a need for modifying the normal workings of the digestive tract to induce weight loss and, thereby, treat obesity without the complications attendant procedures such as Gastric Bypass and gastric banding.

In the normal workings of the digestive tract, food travels from the mouth and then through the esophagus to the stomach. In the stomach, gastric juice is secreted and mixes with the food to produce chyme, i.e., partially digested food. The chyme is then emptied into the small intestine through the pylorus, i.e., the region of the stomach which connects to the duodenum. In the small intestine, chyme mixes with digestive fluids. In particular, in the duodenum (i.e., the upper portion of the small intestine which is proximate to the stomach), chyme mixes with bile (which flows from the common bile duct) and pancreatic fluid (which flows from the pancreatic duct) upon entry of the bile and pancreatic fluid into the duodenum at the papilla of vater (i.e., the point of opening of the common bile duct and pancreatic duct into the duodenum).

As a result of the mixing of chyme with digestive fluids including pancreatic fluid and bile in the small intestine and, more particularly, in the duodenum, chyme is chemically broken down so that nutrients contained therein may be absorbed across the wall of the small intestine. Specifically, carbohydrates are broken down into simple sugars, proteins are broken down into amino acids, and fats are broken down into fatty acids. Pancreatic fluid and, more particularly, the digestive enzymes contained therein, aid(s) in the digestion of proteins, carbohydrates, and fats, while bile aids in the emulsification, digestion, and absorption of fats. Reabsorption of the products of digestion and, more particularly, reabsorption of fatty acids and simple sugars in the duodenum, however, can ultimately lead to the deposition of fat deposits in the body and, consequently, weight gain.

In view of the foregoing, methods which modify the normal workings of the gastrointestinal intestinal system to minimize digestion and absorption of ingested food in the small intestine would be useful for treating individuals who are overweight and, more particularly, individuals who are obese. Specifically, methods which minimize the digestion of fats and carbohydrates and the reabsorption of the products of digestion (particularly fatty acids and simple sugars) in the duodenum, without the complications of known procedures, would be useful for treating individuals who are overweight and, more particularly, individuals who are obese. In particular, methods which may be easier to perform than gastric bypass procedure and that, therefore, decrease procedure time and costs may be useful. The present invention may provide such methods.

### SUMMARY OF THE INVENTION

The present invention provides methods for inducing weight loss in an individual. In particular, the present invention provides methods for inducing weight loss which involve modifying the workings of the gastrointestinal tract by modifying the structure of the duodenum to separate physically chyme from digestive fluids such as bile and pancreatic fluid in the duodenum during the digestive process. In accordance with the present inventive methods, in some embodiments, the duodenum is endosurgically modified to carve out a separate channel within the lumen of the duodenum. In other embodiments, the duodenum is surgically modified to form a circumferential pouch-cuff.

The channel and circumferential pouch-cuff of the present invention may be shaped and oriented in any manner and appropriately sized to slow the digestion of food and/or bile and/or to divert the progression of food and/or bile in the digestive system in a manner which promotes weight loss. In some embodiments, the channel may include a pouch-like formation. Moreover, channel may be of any length. In some embodiments, channel may include a pouch which is of any suitable length.

In some embodiments, channel may be of any suitable length to slow the progression of food through the digestive system to induce weight loss. For example, channel may be of any suitable length to slow the progression of food through the digestive system to slow digestion and induce weight loss.

In other embodiments, channel may be of any suitable length to slow the progression of bile through the digestive system to thereby induce weight loss. For example, channel may be of any suitable length to slow the mixing of chyme with digestive fluids (such as bile) to slow digestion and induce weight loss.

Moreover, in some embodiments, channel is oriented to divert food and/or bile in the digestive system in a manner which induces weight loss. For example, channel may be oriented in such a manner to divert food and/or bile so that the digestive processes are slowed in such a manner that weight loss is induced.

It will be understood that, in some embodiments, the separate channel may partially, substantially or completely prevent the intermixing of chyme with digestive fluids (i.e., bile and pancreatic fluid) in the duodenum, as may the circumferential pouch-cuff. By partially, substantially or completely physically preventing the intermixing of chyme with digestive fluids (i.e., bile and pancreatic fluids) in the duodenum by means of a separate channel formed within the duodenum or by means of a partial or complete circumferential pouch-cuff, the methods of the present invention may reduce the amount of digestion in the duodenum.

In addition, or in the alternative, in some embodiments, the separate channel may simply slow the mixing of chyme with digestive fluids (i.e., bile and pancreatic fluid) in the duodenum, as may the circumferential pouch-cuff. By slowing the mixing of chyme with digestive fluids (i.e., bile and pancreatic fluid) in the duodenum by means of a partial or complete circumferential pouch-cuff, the methods of the present invention may reduce the amount of digestion in the duodenum.

Consequently, the methods of the present invention may reduce the amount of absorption of the products of digestion (such as fatty acids and simple sugars) which occurs in the duodenum. As a result, the amount of weight a person gains from eating a given amount of food may be reduced.

In one aspect of the invention, there is provided a method for inducing weight loss in a patient including the steps of: (i) accessing an intestine having a proximal end, a distal end, an outer surface, and an original lumen extending therethrough; and (ii) bringing intestinal tissue (such as duodenal tissue) from different locations on the intestine into abutting relationship along a length of intestine to form a separate channel within the intestine.

In another aspect of the invention, there is provided a method for inducing weight loss in a patient including the steps of: (i) grasping an intestine having an original lumen across the diameter of the intestine at the distal end of the intestine to bring intestinal tissue (such as duodenal tissue) from different locations on the intestine into contact; (ii) employing a tissue-connecting device to attach the intestinal tissue from the different locations on the intestine; and (iii) continuing to employ the tissue-connecting device to attach intestinal tissue from different locations on the intestine until a location on the outer surface of intestine that is between the papilla of vater and pylorus is reached; wherein steps (i)-(iv) result in the formation of two separated channels within the intestine.

In still another aspect of the invention, there is provided a method for inducing weight loss in a patient including the steps of: (i) positioning a tissue-connecting device at a location between the pylorus and the papilla of vater on the outer surface of a intestine having an original lumen; (ii) employing the tissue-connecting device in a radial direction towards the center of said intestine to join tissue from different locations on the intestine together by angling the tissue-connecting device distally relative to the pylorus and then employing said tissue-connecting device along a length of the intestine until a location between the papilla of vater and Ligament of Treitz is reached; and (iii) continuing to employ the tissue-connecting device along the length of the intestine until a location near the Ligament of Treitz is reached.

In yet another aspect of the invention, there is provided a method for inducing weight loss in a patient including: (i) accessing an intestine having a proximal end, a distal end, and an original lumen extending therethrough; (ii) inserting a medical device into the proximal end of the intestine; (iii) engaging intestinal tissue (such as duodenal tissue) at a location that is between the papilla of vater and the Ligament of Treitz by means of the medical device such that the intestinal tissue becomes attached to the medical device; and (iv) retracting the medical device and the tissue attached thereto to a location within the original lumen that is between the papilla of vater and the pylorus; wherein a circumferential pouch-cuff is formed at the proximal end of the intestine as a result of retracting the endoscopic device.

In yet other embodiments, there is provided a system for inducing weight loss in a patient including:

(a) a grasping means for grasping an intestine and bringing intestinal tissue (such as duodenal tissue) into abutting relationship; and

(b) a connecting device for attaching in abutting relationship tissues to form a common wall which separates an intestine into at least two channels along a portion of a length of the intestine.

### BRIEF DESCRIPTION OF THE DRAWINGS

FIG. 1 is a lengthwise cross-sectional view of the central portion of an alimentary canal including an unmodified duodenum.

FIG. 2 is widthwise cross-sectional view of the distal end of the unmodified duodenum shown in FIG. 1 across line L-L.

FIG. 3 is a widthwise cross-sectional view of the distal end of the unmodified duodenum shown in FIG. 2 after modification in accordance with a method of the present invention.

FIG. 4 is a lengthwise cross-sectional view of the central portion of an alimentary canal including a modified duodenum formed in accordance with the present invention.

FIGS. 5-6 are each perspective views of the central portion of an alimentary canal including a modified duodenum formed in accordance with the present invention using a tissue-connecting device.

FIG. 7 is a cross-sectional lengthwise view of the central portion of an alimentary canal including a duodenum having a medical device positioned therein prior to retraction of the medical device in accordance with a method of the present invention.

FIG. 8 is a perspective view of the alimentary canal shown in FIG. 7 having a supporting member positioned on duodenum in accordance with a method of the present invention.

FIG. 9 is a cross-sectional lengthwise view of the alimentary canal shown in FIGS. 7 and 8 having a medical device retracted therein to form a modified duodenum having a circumferential pouch-cuff in accordance with a method of the present invention.

FIG. 10 is a perspective lengthwise view of the alimentary canal including a modified duodenum having a circumferential pouch-cuff shown in FIG. 9.

FIG. 11 is a lengthwise cross-sectional view of the alimentary canal shown in FIG. 10 where needles are used to secure the circumferential pouch-cuff in place.

FIG. 12 is a widthwise cross-sectional view of the alimentary canal shown in FIG. 11 along line A-A.

FIG. 13 is a cross-sectional lengthwise view of a section of an alimentary canal including a duodenum having a medical device including a suction device positioned therein in accordance with a method of the present invention.

FIG. 14 is a perspective view of the alimentary canal shown in FIG. 13 after medical device including suction device is retracted to form a modified duodenum having a circumferential pouch-cuff in accordance with a method of the present invention.

FIG. 15 is a cross-sectional lengthwise view of the alimentary canal shown in FIG. 14 including modified duodenum having circumferential pouch-cuff after the deployment of needles on the suction device and after the placement of a ring-shaped member over the needles in accordance with a method of the present invention.

FIG. 16 is a cross-sectional lengthwise view of the alimentary canal shown in FIG. 14 including modified duodenum having circumferential pouch-cuff after the deployment of needles on the suction device and after the placement of caps on the ends of the needles in accordance with a method of the present invention.

FIG. 17 is an exploded cross-sectional view of the alimentary canal including modified duodenum shown in FIG. 15 along line B-B.

FIG. 18 is an exploded cross-sectional view of the alimentary canal including modified duodenum shown in FIG. 16 along line C-C.

FIG. 19 is a perspective view of the alimentary canal including modified duodenum shown in FIG. 15.

FIG. 20 is a perspective view of the alimentary canal including modified duodenum shown in FIG. 16.

FIG. 21 is a cross-sectional view of the alimentary canal including modified duodenum shown in FIGS. 15 and 19 illustrating how circumferential pouch-cuff prevents or substantially prevents the intermixing of chyme with digestive fluids.

FIG. 22 is a cross-sectional view of the alimentary canal including modified duodenum shown in FIGS. 16 and 20 illustrating how circumferential pouch-cuff prevents or substantially prevents the intermixing of chyme with digestive fluids.

FIG. 23 is a perspective view of a tissue-connecting device for use in the invention.

FIG. 24 is a top view of a strip having a combination of fasteners thereon.

FIG. 25 is a widthwise cross-sectional view of the distal end of a modified duodenum of the invention having strips with fasteners thereon.

FIG. 26 is a side view of a fastener for use in the invention.

FIG. 27 is an exploded widthwise cross-sectional view of the distal end of a modified duodenum of the invention having multiple layers formed from multiple folds of one or more than one section of the intestine.

### DETAILED DESCRIPTION OF THE INVENTION

In some embodiments, this invention provides methods, such as endosurgical methods, to partially, substantially or completely separate chyme from bile and pancreatic fluids in the small intestine and, more specifically, in the duodenum. In other embodiments, this invention provides methods, such as endosurgical methods, to slow the mixing of chyme with bile and pancreatic fluids in the small intestine and, more specifically, in the duodenum. Such methods may promote weight loss by delaying or preventing lipid and carbohydrate breakdown and absorption in the small intestine and, more specifically, in the duodenum. In particular, this invention provides methods of separating chyme from digestive fluids (particularly, bile and pancreatic fluids) partially, substantially or completely in the small intestine and, more specifically, in the duodenum. As a result, chyme desirably does not mix, or mixes to a lesser extent, or mixes more slowly with the digestive fluids (i.e., bile and pancreatic fluid) along at least a portion of the length of the duodenum. In some embodiments, chyme desirably does not mix, or mixes to a lesser extent, or mixes more slowly with the digestive fluids (i.e., bile and pancreatic fluid) along a portion of the duodenum. In some embodiments, chyme desirably does not mix, or mixes to a lesser extent, or mixes more slowly with the digestive fluids (i.e., bile and pancreatic fluid) along a substantial portion of the duodenum.

By partially, substantially or completely separating chyme from digestive fluids (i.e., bile and pancreatic fluid) in the small intestine, and, more particularly, in the duodenum, it may be possible to reduce the amount of digestion. Consequently, it may be possible to reduce the amount of reabsorption of the products of digestion (such as fatty acids and simple sugars) which occurs in the small intestine. Fatty acids and simple sugars which are absorbed across the wall of the duodenum are often deposited in the body as fat if not immediately needed to meet the metabolic requirements of the body. Therefore, by decreasing digestion of fats and carbohydrates and/or by promoting malabsorption of fatty acids and simple sugars, it may be possible to induce weight loss. Likewise, by slowing the digestion of fats and carbohydrates and/or by slowing the absorption of fatty acids and simple sugars, it may be possible to induce weight loss. Accordingly, the methods of the present invention desirably promote weight loss and treat obesity by decreasing and/or slowing digestion of ingested food (particularly, by decreasing digestion of fats and carbohydrates) in the small intestine (particularly, the duodenum). Such methods also desirably promote weight loss and treat obesity by consequently promoting malabsorption and/or slowing absorption of the products of digestion (such as fatty acids and simple sugars) in the small intestine (particularly, in the duodenum).

In addition, or in the alternative, in some embodiments, by causing chyme to mix to a lesser extent, or to mix more slowly with the digestive fluids (i.e., bile and pancreatic fluid) along the length of the duodenum or a portion thereof, it may be possible to decrease the rate of breakdown of chyme and, consequently, the rate of absorption of the byproducts of digestion. Moreover, by decreasing the rate of breakdown of chyme, an individual may feel satiated for a longer period of time, thereby causing a person not to consume as many calories over a given period of time. As a result of the possibility of decreased absorption at any given time and as a result of the possibility of increased feelings of satiation for longer periods, there may be lesser opportunity for fatty acids and simple sugars to be deposited in the body as fat. Accordingly, weight loss may ensue.

These and other features of the invention will be more fully understood from the following description of specific embodiments of the invention taken together with the accompanying drawings. Unless otherwise defined herein, it should be noted that references herein to the term “distal” are to a direction towards the distal end of the duodenum, while references to the term “proximal” are to a direction towards the proximal end of the duodenum.

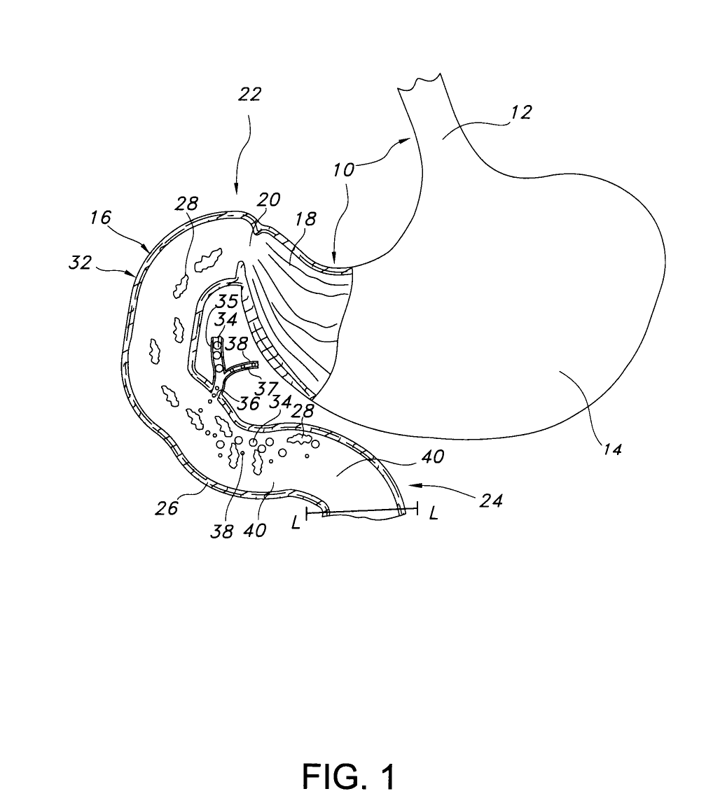

Referring to the drawings and, more particularly to FIG. 1, a lengthwise cross-sectional view of the central portion of an alimentary canal 10 is illustrated. This portion of the alimentary canal 10 includes the distal segment of the esophagus 12, the stomach 14, and the duodenum 16. The duodenum 16 is the proximate segment of the small intestine relative to the stomach 14, and has a proximal end 22 and a distal end 24. The stomach 14 has a pyloric portion 18 which leads to the duodenum 16 by way of the gastric outlet or pylorus 20.

Chyme 28 passes from the pyloric portion 18 through the pylorus 20 into the duodenum 16. The duodenum 16 has an inner surface 26, an outer surface 32, and an original lumen 40. Digestive fluids (i.e., bile 34 flowing from bile duct 35 and pancreatic fluid 38 flowing from pancreatic duct 37) flow through the papilla of vater 36 into the lumen 40 of the duodenum 16. As illustrated in FIG. 1, in the normal workings of the gastrointestinal system, chyme 28 mixes with digestive fluid (i.e., bile 34 and pancreatic fluid 38) in the original lumen 40 of duodenum 16. As a result of chyme 28 mixing with the digestive fluids (i.e., bile 34 and pancreatic fluid 38), digestion ensues and chyme 28 is chemically broken down. Any fatty acids and simple sugars resulting from the chemical breakdown of the chyme 28 then may be reabsorbed along the intestinal wall of the duodenum 16 and subsequently deposited in the body as fat if not needed by the body.

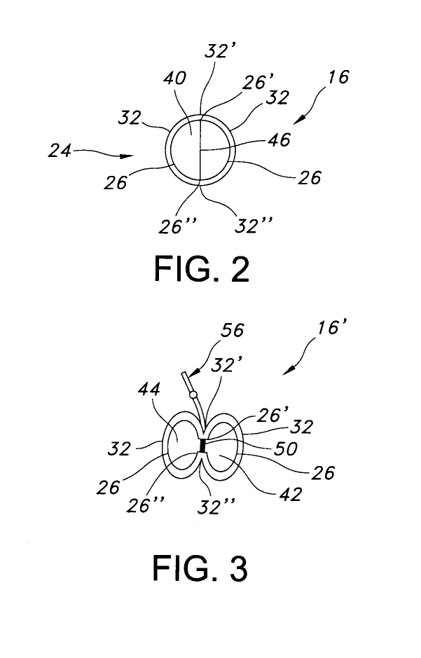

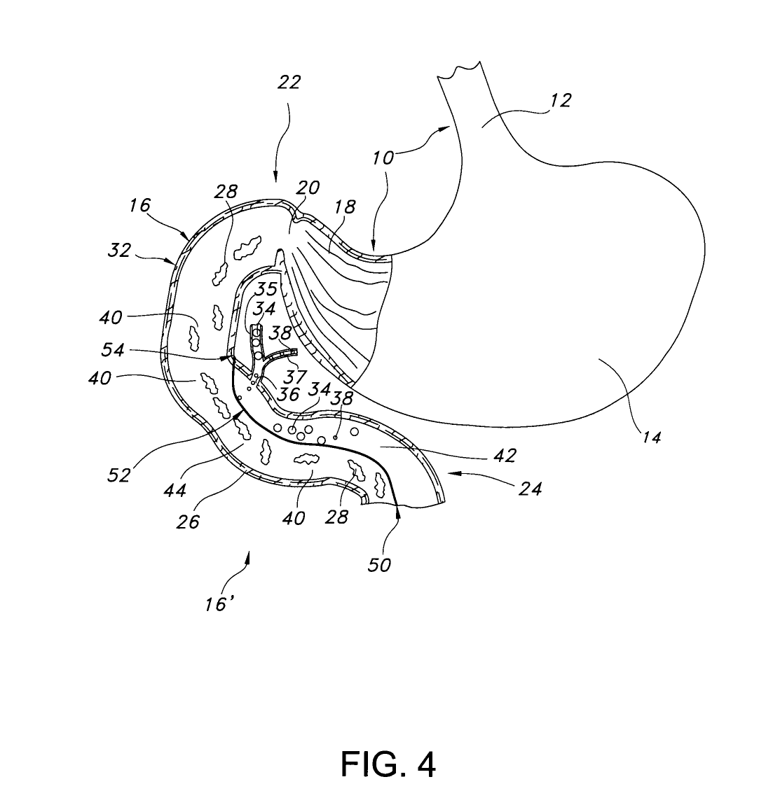

In one embodiment of the invention, a method for inducing weight loss in a patient includes accessing a duodenum 16 as illustrated in FIG. 1. Duodenum 16 has a proximal end 22, a distal end 24, an outer surface 32, an inner surface 26, and an original lumen 40 extending therethrough. In particular, duodenal tissue from different locations 26′, 26″ on the inner surface 26 of duodenum 16 as shown in FIG. 2 (which is a widthwise cross-sectional view of the distal end 24 of duodenum 16 shown in FIG. 1 across line L-L) is brought into contact and attached to form a modified duodenum 16′ having a separate channel 42 within lumen 40. More particularly, two separate channels 42, 44 are formed out of original lumen 40 to form a modified duodenum 16′ having a widthwise cross-sectional view along at least a length thereof as shown in FIG. 3 and a lengthwise cross-sectional view as show in FIG. 4.

Separate channels 42 and 44 share a common wall 50 which, in some embodiments, may substantially separate original lumen 40 of duodenum 16 along at least a length thereof, as illustrated in FIG. 3. Moreover, as illustrated in FIG. 4, common wall 50 includes a common line of tissue engagement 52 which may be formed in some embodiments by bringing duodenal tissue from different locations 26′, 26″ which span a length of duodenum 16 on the inner surface 26 of duodenum 16 into contact along a length of duodenum 16 as described above. As illustrated in FIG. 4, common line 52 of tissue engagement may extend from the distal end 24 of the duodenum to a location 54 on the outer surface 32 of duodenum 16 which is between the pylorus 20 and the papilla of vater 36.

In some embodiments, the duodenal tissue from different locations 26′, 26″ on the inner surface 26 of duodenum 16 may be brought into abutting relationship. This may be accomplished by grasping the duodenum 16 shown in FIG. 1 about its outer surface 32 and then attaching the duodenal tissue from different locations 26′, 26″ to form the common line 52 of tissue engagement of the modified duodenum 16′ shown in FIG. 4. Any tissue-connecting device may be used to grasp duodenum 16, bring the duodenal tissue into abutting relationship, and attach it together as described.

As shown in FIG. 4, channel 42 receives digestive fluids (i.e., bile fluid 34 and pancreatic fluid 38) flowing into the modified duodenum 16″ from the papilla of vater 36, while channel 44 receives chyme 28 flowing from the pylorus 20. As common wall 50 is impermeable or substantially impermeable to digestive fluids (i.e., bile 34 and pancreatic fluid 38), chyme 28 is desirably partially or completely prevented from mixing with the digestive fluids (i.e., bile 34 and pancreatic fluid 38). In some embodiments, wall 50 may simply slow down the mixing of digestive fluid with food.

Due to the partial or complete separation of chyme 28 from digestive fluids (i.e., bile 34 and pancreatic fluid 38) and/or the slowing down of the mixing of digestive fluid with food, the bile 34 may be prevented and/or slowed from breaking down fats in the chyme 28. Moreover, the pancreatic fluid 38 may be prevented from aiding in the digestion of carbohydrates, fats, and proteins and/or may slow the digestion of the carbohydrates, fats, and proteins. Accordingly, the amount of digestion and the amount of reabsorption of the products of digestion (such as fatty acids and simple sugars) in the modified duodenum 16′ may be reduced, thereby reducing the amount of weight a person gains from eating a given amount of food and consequently inducing weight loss. In particular, fats may not be broken down as completely and reabsorbed as completely in the modified duodenum 16′ as in the normal workings of the gastrointestinal tract. This is because bile 34 may be substantially prevented from mixing with fats in the chyme 28. Moreover, bile 34 may be reabsorbed in the modified duodenum 16′ so that it is not available to react with fat in the chyme 28 both while the chyme 28 is in the modified duodenum 16′ and after it passes into the rest of the small intestine (not shown). As a result, weight loss may be induced.

In some embodiments, a method for inducing weight loss in a patient includes the step of grasping the outer surface 32 of the distal end 24 of a duodenum 16 as illustrated in FIG. 1 across the diameter 46 of the distal end 24 of the duodenum 16, as shown in FIG. 2. Duodenal tissue from different locations 26′ and 26″ on the inner surface 26 of duodenum 16 is then brought into contact as illustrated in FIG. 3, which is a widthwise cross-sectional view of the distal end of the duodenum 16 shown in FIG. 2 after such modification. In particular, duodenal tissue on different locations 32′, 32″ on the outer surface of duodenum 32 and, desirably, duodenal tissue from opposing locations, on the outer surface 32 of duodenum 16 is grasped. The duodenal tissue 32′, 32″ may be grasped such that corresponding duodenal tissue from different locations 26′, 26″ on the inner surface 26 of duodenum 16 may be brought into abutting relationship. As a result, a modified duodenum 16′ having a widthwise cross-sectional view as illustrated in FIG. 3 and a cross-sectional lengthwise view as illustrated in FIG. 4 may be formed.

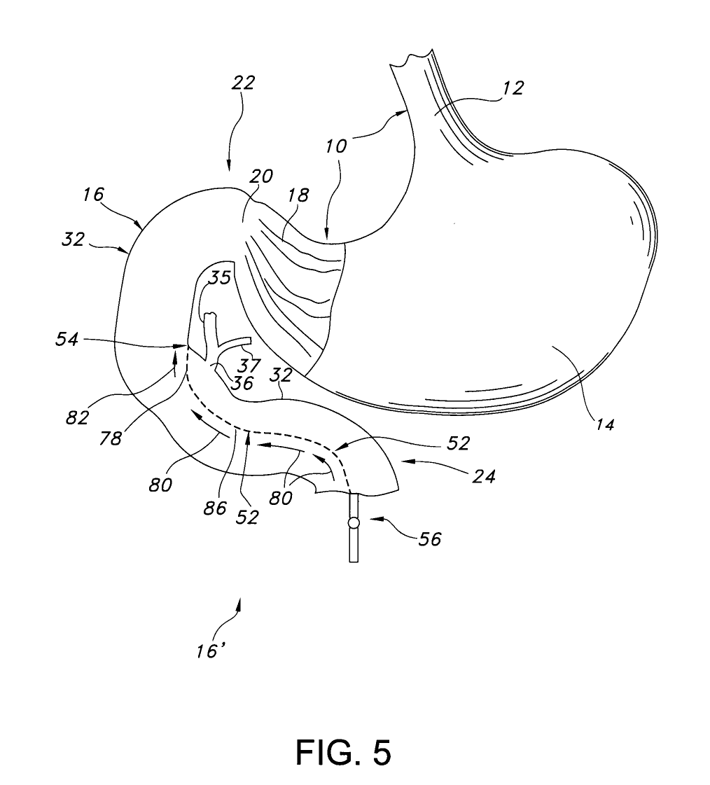

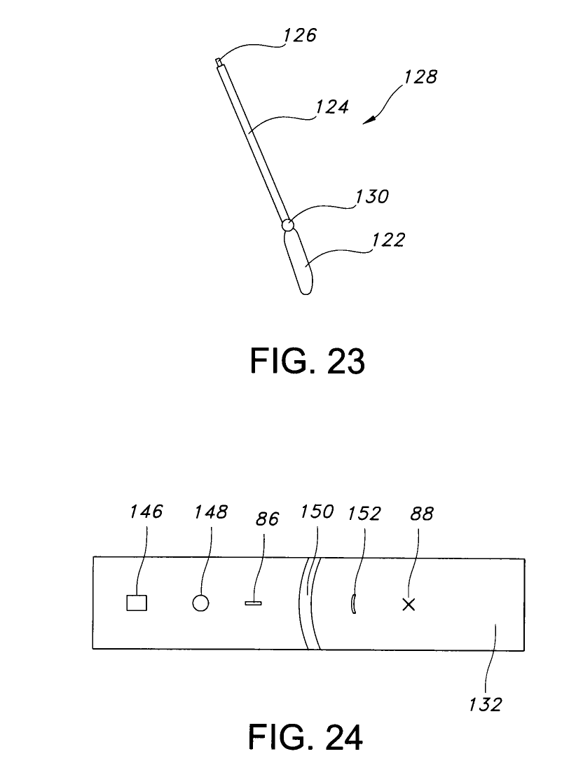

In such embodiments, a tissue-connecting device as illustrated in FIG. 23 may be used. In particular, as illustrated in FIGS. 3 and 5, tissue-connecting device 56 may be applied to the outer surface 32 of duodenum 16. Tissue-connecting device 56 may be employed such that duodenal tissue from different locations 26′, 26″ on the inner surface 26 of the distal end 24 of duodenum 16 is secured together. In particular, as shown in the perspective view of FIG. 5, the tissue-connecting device 56 may be employed continuously along at least a portion of the length of the outer surface 32 of duodenum 16 in the direction of the arrows 80 shown in FIG. 5. By employing tissue-connecting device 56 in this manner, duodenal tissue from different locations 26′, 26″ on inner surface 26 (not shown in FIG. 5) may be brought into contact along a length of the duodenum 16 until a location 78 within the original lumen 40 is reached. Desirably, location 78 is between the papilla of vater 36 and pylorus 20.

Thereafter, tissue-connecting device 56 may be continuously employed radially above the papilla of vater 36 in the direction of the arrow 82 as shown in FIG. 5. As a result, tissue from different locations 26′, 26″ on the inner surface 26 of duodenum 16 may be attached until a location 54 on the outer surface 32 of duodenum 16 is reached. Location 54 is desirably between the papilla of vater 36 and pylorus 20. By such a method, two distinct channels 42, 44 may be formed within the original lumen 40 of duodenum 16 to form a modified duodenum 16′. Modified duodenum 16′ has a widthwise cross-sectional view along at least a length thereof as illustrated in FIG. 3. As illustrated in FIG. 3, in some embodiments, separate channels 42 and 44 share a common wall 50 which substantially separates original lumen 40 along at least a length thereof.

Although it is the duodenal tissue from different locations 26′, 26″ on the inner surface 26 of the distal end 24 of duodenum 16 that may be brought into contact, it will be understood that the tissue-connecting device 56 may be employed external to the duodenum 16. In particular, by externally clamping duodenum 16 along a length thereof by means of the tissue-connecting device 56, duodenal tissue from different locations 26′, 26″ on the inner surface 26 spanning a length of duodenum 16 may be brought into contact and attached to form channels 42 and 44.

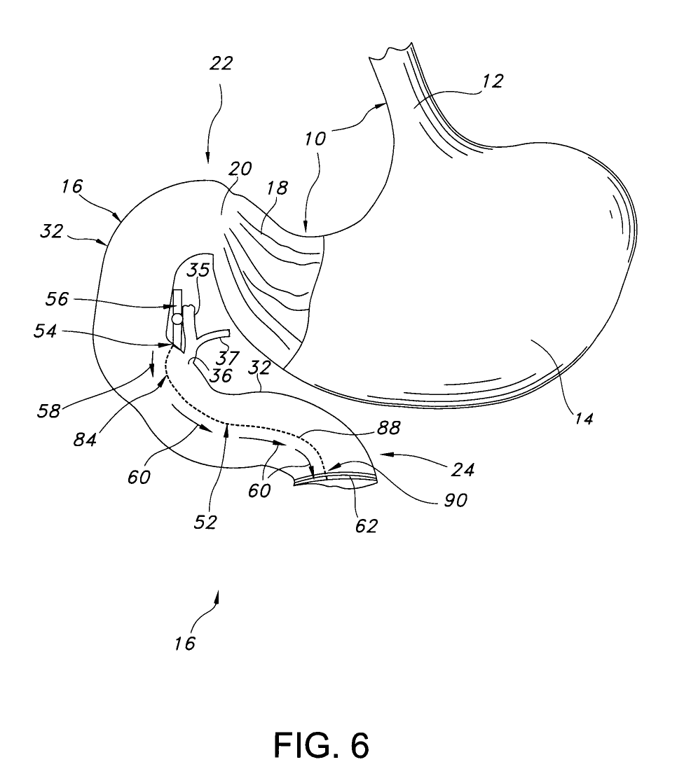

In another aspect of the invention, a method for inducing weight loss in a patient includes positioning a tissue-connecting device 56 at a location 54 between the pylorus 20 and the papilla of vater 36 on the outer surface 32 of duodenum 16 as illustrated in FIG. 6, which is a perspective view of a modified duodenum 16′ in accordance with the present invention. The tissue-connecting device 56 may then be employed in a radial direction in the direction of the arrows 58 towards the center of the lumen 40 of the duodenum 16 to join tissue from different locations 26′, 26″ on the inner surface 26 of the duodenum 16 (not shown in FIG. 6). In particular, tissue-connecting device 56 may be angled distally relative to the pylorus until an initial location 84 within the original lumen 40 of duodenum 16 is reached. Desirably, initial location 84 is between the papilla of vater 36 and the Ligament of Treitz 62, as illustrated in FIG. 6. Thereafter, tissue-connecting device 56 may be continuously employed distally in the direction of the arrows 60 along the length of duodenum 16. In particular, tissue-connecting device 56 may be employed until a location 90 which is near the Ligament of Treitz 62, i.e., the suspensory ligament which connects the duodenum 16 to the diaphragm (not shown) is reached.

As a result, at least two channels 42, 44 can be formed within the original lumen 40 of the duodenum 16 to form a modified duodenum 16′ having a lengthwise cross-sectional view as illustrated in FIG. 4 and a perspective view as illustrated in FIG. 5. As further illustrated in FIG. 4, common wall 50 may include a line of tissue engagement. Line of tissue engagement may be formed, in some embodiments, by bringing duodenal tissue from locations 26′, 26″ which span a length of duodenum 16 on the inner surface 26 of duodenum 16 into contact along a length of duodenum 16 as described above. As illustrated in FIG. 4, common line of tissue engagement may extend from the distal end 24 of the duodenum 16 to a location 54 on the outer surface of duodenum 16 which is between the pylorus 20 and the papilla of vater 36. In some embodiments, separate channels 42 and 44 may share a common wall 50 which, in some embodiments, may separate original lumen 40 of duodenum 16 along at least a length thereof.

Location 90 may be any suitable distance from the Ligament of Treitz. Desirably, location 90 is about 10 cm from the Ligament of Treitz.

As illustrated in FIG. 4, common wall 50 may result in the substantial separation of digestive fluids (i.e., bile 34 and pancreatic fluid 38) from chyme 28. Consequently, channel 42 can receive digestive fluids (i.e., bile 34 and pancreatic fluid 38) flowing into the modified duodenum 16′ from the papilla of vater 36, while channel 44 can receive chyme 28 flowing from the pylorus 20. As common wall 50 may substantially prevent, impede, and/or slow the mixing of digestive fluids (e.g., bile 34 and pancreatic fluid 38) with chyme 28 and/or partially separates chyme 28 from digestive fluids (e.g., bile 34 and pancreatic fluid 38), the mixing of the digestive fluids with chyme 28 may be substantially prevented, impeded, and/or slowed. As a result, the amount of digestion of chyme 28 and the amount of reabsorption of digestion byproducts such as fatty acids and simple sugars in the modified duodenum 16′ may be reduced. In particular, fats may not be broken down and reabsorbed in the modified duodenum 16′ as in the normal workings of the gastrointestinal tract because bile 34 may be substantially prevented, impeded, and/or slowed from mixing with fats in the chyme 28. Moreover, bile 34 may be reabsorbed in the modified duodenum 16′ so that it is not available to react with fat in the chyme 28 both while the chyme 28 is in the modified duodenum 16′ and after it passes into the rest of the small intestine (not shown). Thus, the amount of weight a person gains from eating a given amount of food may be reduced. As a result, weight loss may be induced.

With respect to all of the above embodiments, it will be understood that, depending on the type of tissue-connecting device 56 used, common line 52 of tissue engagement may include staples 86 as shown in FIG. 5 or sutures or stitches 88 as illustrated in FIG. 6.

Tissue-connecting device 56 may be any suitable medical device including a means for connecting tissue or at least one component for connecting tissue. For example, tissue-connecting device 56 may be a stapler, such as a linear stapler which does not cut tissue, or a stitch or suture-imparting device. When tissue-connecting device 56 comprises a laparoscopic stapler, common line 52 of tissue engagement will include a staple line having staples 86 as shown in FIG. 5. When tissue-connecting device 56 comprises a stitch or suture-imparting device, common line 52 of tissue engagement will comprise a suture line having sutures or stitches 88 as shown in FIG. 6.

Moreover, it will be understood that tissue-connecting device 56 may be any device capable of delivering at least one tissue-connecting means 126, which may be temporary and/or removable. As illustrated in FIG. 23, tissue-connecting device 56 may be, for example, a device 128 which includes a handle 122, at least one component 124 which is capable of delivering at least one tissue-connecting means 126 to intestinal and, more particularly, duodenal tissue in accordance with the invention, and at least one component 130 which connects the handle 122 to the at least one component 124 which is capable of delivering at least one tissue connecting means 126.

Tissue connecting means 126 may include, for example, anchors, fasteners, staples, cuffs, clips and/or sutures any/or any combination thereof. In particular, in some embodiments, anchors, cuffs, fasteners, staples, clips, and/or sutures may be placed at various locations on the inner surface 26 of duodenum 16, on the outer surface 32 of duodenum 16, or on both the inner surface 26 and outer surface 32 of duodenum 16. In particular, the anchors, fasteners, staples, cuffs, clips and/or sutures any/or any combination thereof may be temporary and/or removable.

In some embodiments, wall 50 may be formed with a series of anchors 146, fasteners 138, staples 85, cuffs 150, clips 152, and/or combinations thereof and/or with a strip 132 including these mechanisms and/or capable of delivering these mechanisms to duodenal or intestinal tissue. A top planar view of a strip 132 including a combination of the aforementioned mechanisms is illustrated in FIG. 24.

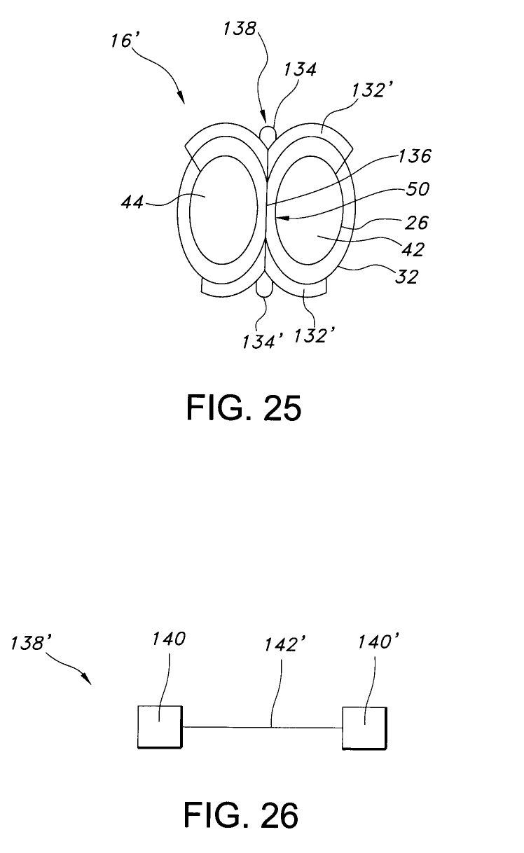

Strip 132 may have any suitable shape and, more specifically, may be shaped to improve the seal between the intestinal and, more particularly, the duodenal tissues 26′, 26″ when common wall 50 is formed. In particular, strip 132 may be made of any suitable material which may conform to the outer surface 32 and/or inner surface 26 of a duodenum 16 after common wall 50 and separate channels 42 and 44 are formed. In some embodiments, strip 132 may be a strip 132′ having a convex shape, as illustrated in FIG. 25, which is a widthwise cross-sectional view of the distal end of a modified duodenum 16′ having separate channels 42 and 44 and a common wall 50 in accordance with a method of the present invention.

Moreover, as illustrated in FIG. 25, more than one strip 132, 132′ may be placed on the outer surface 32 of duodenum 16. In some embodiments, as further illustrated in FIG. 25, a fastener 138, such as a T fastener having two heads 134, 134′ connected with a filament 136, may adhere strips 132, 132′ to the outer surface 32 of duodenum 16. Heads 134, 134′ may be of any suitable shape. As illustrated in FIG. 25, heads 134, 134′ may be buttons. In some embodiments, fastener may be a T fastener 138′ having two rectangular heads 140, 140′ connected by a filament 142′, as illustrated in FIG. 26. Although not illustrated, it will be understood that a strip 132, 132′ may be placed on the inner surface 26 of a modified duodenum 16′ along common wall 50. Moreover, although not illustrated, it also will be understood that a strip 132, 132′ may be placed on both the inner surface 26 of modified duodenum 16′ along common wall 50 and on the outer surface 32 of duodenum 16. Furthermore, a strip 132, 132′ may include at least one male component which can engage a female component on the same strip or on opposite strips.

Moreover, in some embodiments, a strip 132, 132′ may be shaped to improve the seal between the duodenal tissues. For example, in some embodiments, strip 132, 132′ may be shaped to conform to the shape of the intestinal tissue and, more particularly, to the shape of the inner 26 and/or outer 32 surface of duodenal tissue of a modified duodenum 16′ of the invention.

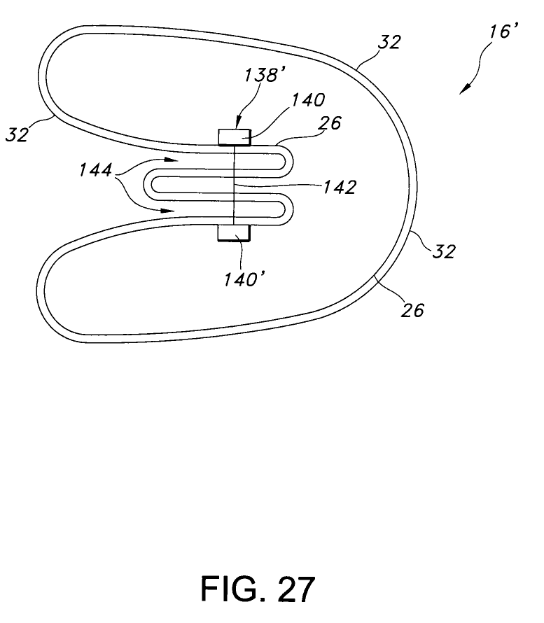

Furthermore, in some embodiments, a common wall 50′ of a modified duodenum 16′ of the invention may include multiple layers formed from multiple folds 144 of one or more section(s) of the intestine, as illustrated in the exploded widthwise cross-sectional view of the distal end of a modified duodenum of the invention having multiple layers formed from multiple folds 144 illustrated in FIG. 27.

As further illustrated in FIG. 27, a fastener such as, for example, a T-fastener 138′ as shown in FIG. 26, may be used to secure the multiple layers formed from multiple folds 144. Although not shown, it may be appreciated that T-fastener 138′ may be positioned so it that extends through the multiple layers formed from multiple folds 144 from one side of the outer surface 32 of a modified duodenum 16′ to an opposite side of the outer surface 32 of a modified duodenum 16′.

In some embodiments, there is provided a system for inducing weight loss in a patient including: (a) a grasping means for grasping an intestine and bringing intestinal tissue (such as duodenal tissue) into abutting relationship; and (b) a connecting device for attaching in abutting relationship tissues to form a common wall which separates an intestine into at least two channels along a portion of a length of the intestine. By “grasping means” is meant any device which can grasp the opposing outer walls of the intestine to form a common wall 50 which separates at least two separate channels 42,44 along a portion of the length of intestine, as illustrated in FIG. 3. Any type of forceps or other common device which may permit the grasping and clamping of the outer surface may be useful. By “connecting device” is meant any means which is capable of connecting intestinal tissue (such as duodenal tissue) along a portion of the length of an intestine. Suitable connecting devices include, for example, any of the tissue-connecting means described herein as well as any other tissue-connecting means known in the art.

With respect to all of the above embodiments, it will be understood that any suitable medical means known in the art may be used to facilitate the placement of the tissue-connecting device 56 on duodenum 16. For example, endoscopic visualization may be used to facilitate the placement of tissue-connecting device 56 on duodenum 16. In particular, endoscopic visualization may be used to identify the location 54 on outer surface 32 of duodenum 16 which is between the pylorus 20 and the papilla of vater 36. Other forms of visualization include, but are not limited to, x-rays, magnetic resonance imaging, and ultrasound.

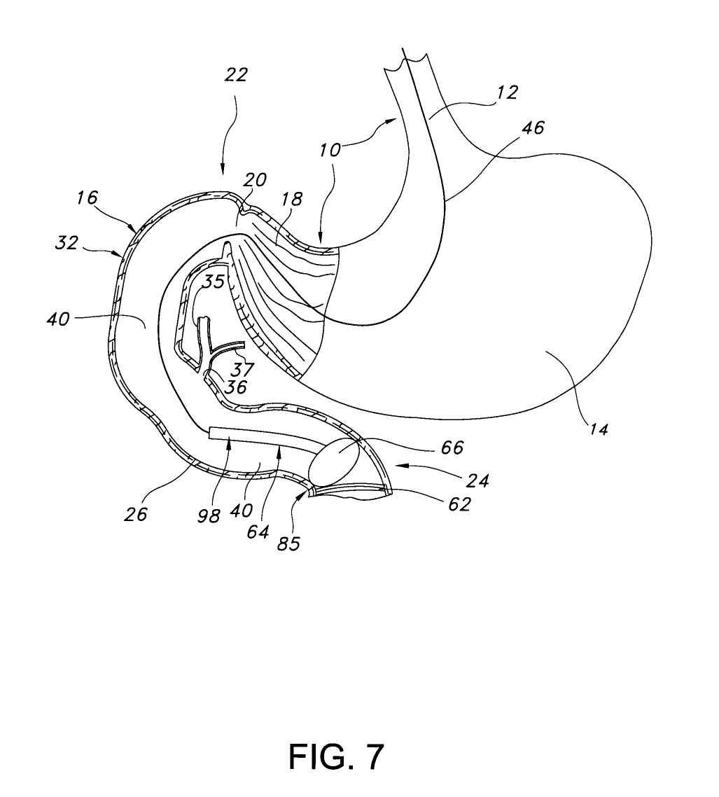

In still another embodiment, a method of inducing weight loss in a patient includes accessing a duodenum 16 having a proximal end 22, a distal end 24, and an original lumen 40 extending therethrough as shown in FIG. 1. In particular, a medical device 64 such as an endoscopic device may be inserted into the proximal end 22 of duodenum 16 using, for example, a guide wire 46 which may be inserted in the alimentary canal 10, as illustrated in FIG. 7, which is a cross-sectional view of the central portion of an alimentary canal 10 including the esophagus 12, stomach 14, and duodenum 16 along a length thereof.

As illustrated in the cross-sectional view of FIG. 7, the medical device 64 may be positioned such that it engages duodenal tissue on the inner surface 26 of duodenum 16 about a circumferential length of duodenum 16 at a location 85 that is between the papilla of vater 36 and the Ligament of Treitz 62.

Location 85 may be any suitable distance distal to the papilla of vater. Desirably, location 85 is several inches distal to the papilla of vater and near the Ligament of Treitz 62, as illustrated in FIG. 7.

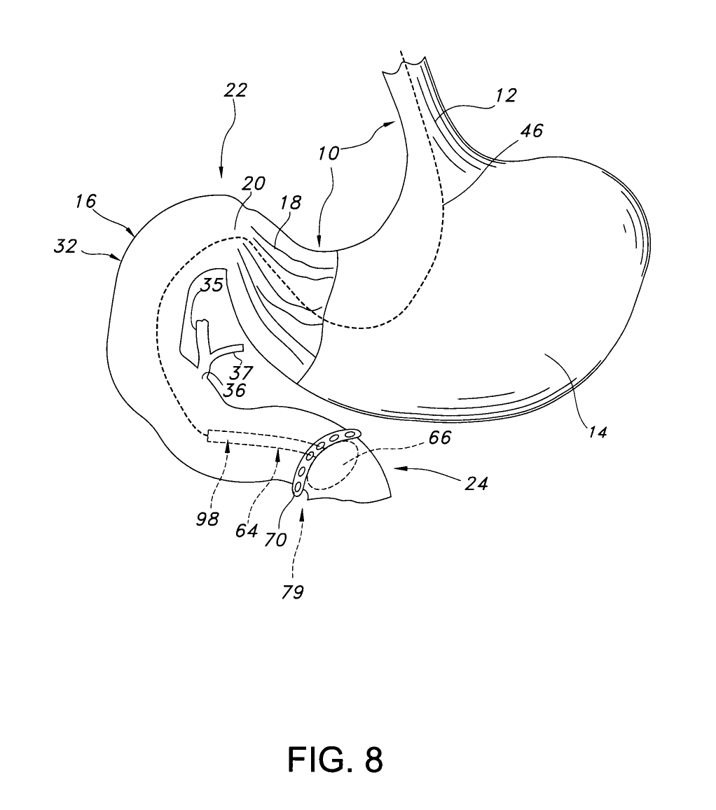

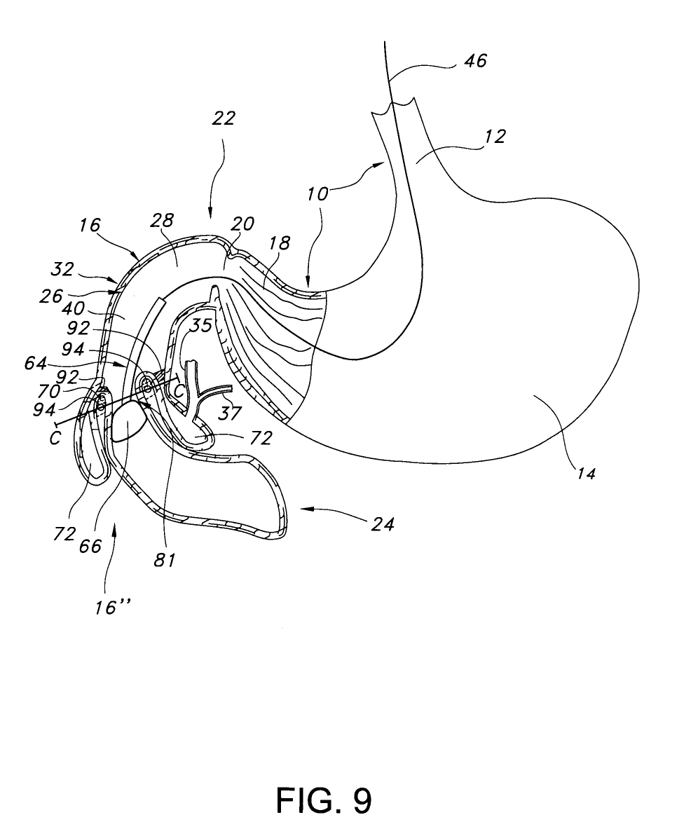

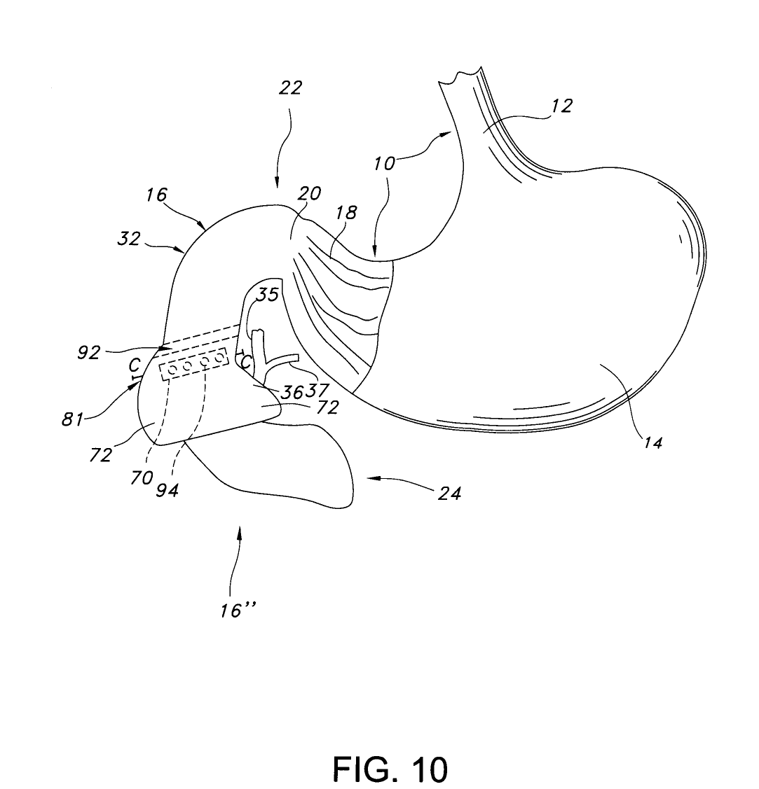

Thereafter, as illustrated in FIG. 8, a supporting member 70 such as a ring-shaped member may be inserted on the outer surface 32 of the duodenum 16 around at least a portion of the circumference of duodenum 16 and, in some embodiments, at or about the location where duodenal tissue becomes attached to the medical device 64. After supporting member 70 is positioned on the outer surface 32 of duodenum 16 as illustrated in FIG. 8, the medical device 64 may be retracted to a location 81 within the original lumen 40 of duodenum 16 that lies at a point between the papilla of vater 36 and the pylorus 20, as illustrated in the lengthwise cross-sectional view of modified duodenum 16″ shown in FIG. 9. In particular, as illustrated in FIGS. 9 and 10, as a result of retracting the medical device 64 in such a manner, the supporting member 70 may be positioned external to the original duodenum 16 but internal to a circumferential pouch-cuff 72, which is a substantially circumferential fold of duodenal tissue formed as a result of the retracting step. With reference to FIG. 10, it will be understood that supporting member 70 (shown in hatch marks) may be positioned under circumferential pouch-cuff 72 but external to original duodenum 16 to form modified duodenum 16″. Guide wire 46 and medical device 64 can then be removed from alimentary canal 10.

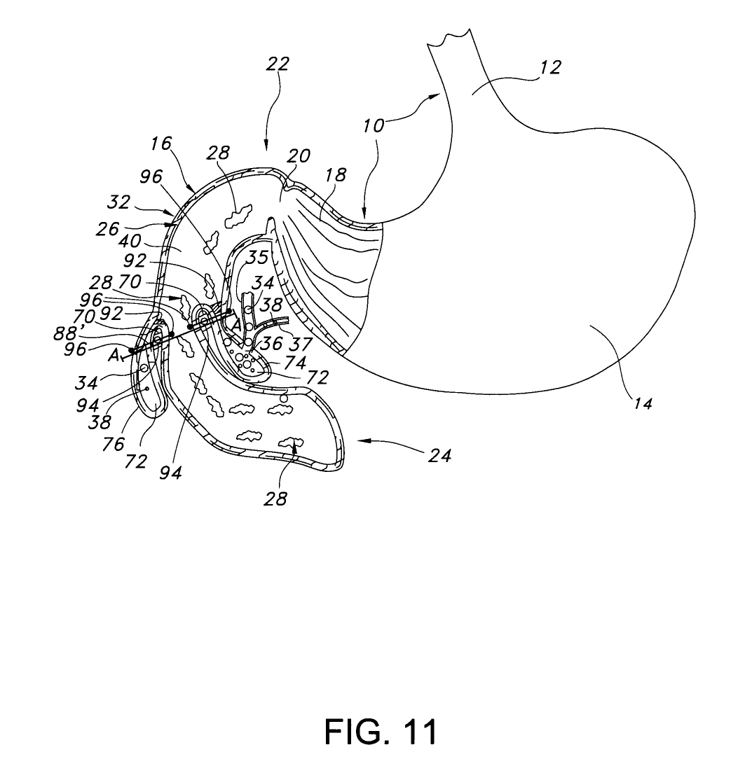

Thereafter, as further illustrated in FIGS. 9 and 10, an anchor 92, such as an annular anchor, can be positioned on the inner surface 26 of duodenum 16 at or near the top of the circumferential fold of duodenal tissue which forms the circumferential pouch-cuff 72. With reference to FIG. 10, it will be understood that annular anchor (shown in hatch marks) may be positioned within the inner surface 26 of duodenum 16 at or near the top of the circumferential fold of duodenal tissue which forms the circumferential pouch-cuff 72. Anchor 92 may then be activated such that chyme 28 flowing from the stomach 14 may pass into the original lumen 40 of duodenum 16 while bile fluid 34 is prevented from flowing into the original lumen 40 of duodenum 16 and mixing with chyme 28, as illustrated in the lengthwise cross-sectional view of modified duodenum 16″ illustrated in FIG. 11. In particular, anchor 92 may be positioned or manipulated such that digestive fluids (i.e., bile 34 and pancreatic fluid 38) flowing through the papilla of vater 36 becomes partially trapped in the circumferential pouch-cuff 72 and significantly prevented from mixing with chyme 28 which flows from the stomach 14, as illustrated in FIG. 11. In particular, anchor 92 may optionally be diametrically adjusted and can be moved along a length of the circumferential pouch-cuff 72 to permit the circumferential pouch-cuff to be of different lengths and volumes. The digestive fluids (i.e., bile 34 and pancreatic fluid 38) which are trapped in circumferential pouch-cuff 72 can then be reabsorbed through the wall 74 of the circumferential pouch-cuff 72.

Due to the separation of chyme 28 from digestive fluids (i.e., bile 34 and pancreatic fluid 38), the amount of digestion of chyme 28 and the amount of reabsorption of digestion byproducts such as fatty acids and simple sugars in the modified duodenum 16″ may be reduced, thereby reducing the amount of weight a person can gain from eating a given amount of food and inducing weight loss. In particular, fats may not be broken down and reabsorbed in the modified duodenum 16″ as in the normal workings of the gastrointestinal tract because modified duodenum 16″ substantially separates bile 34 from chyme 28. As a result, bile 34 is substantially prevented from mixing with fats in the chyme 28 and/or mixing of bile 34 with chyme 28 is slowed. Moreover, bile 34 is reabsorbed in the modified duodenum 16″ so that it is not totally available to react with fat in the chyme 28 both while the chyme 28 is in the modified duodenum 16″ and after it passes into the rest of the small intestine (not shown). As a result, weight loss can be induced.

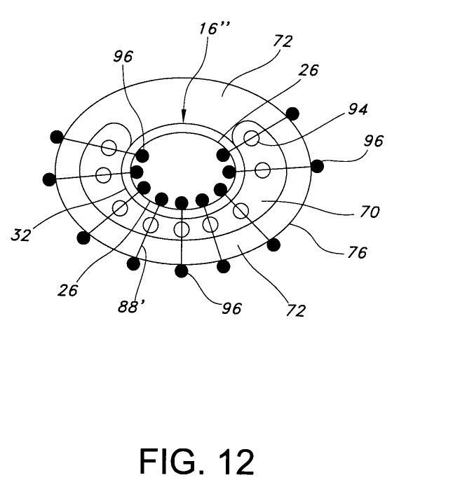

With reference to FIGS. 8 to 11, any suitable supporting member 70 may be employed. Desirably, in some embodiments, supporting member 70 may be a ring-shaped member and, more particularly, a C-shaped plastic ring as illustrated in FIG. 12, which is a top cross-sectional view of modified duodenum 16″ illustrated in FIGS. 9 and 10 along line C-C at location 81 where the supporting member 70 is located after retraction of the medical device 64 as described above.

When a supporting member 70 is used as described above, the supporting member 70 may include at least one hole 94 and, more desirably, a plurality of holes 94 around its surface as illustrated in FIGS. 8 and 12. For example, as illustrated in FIG. 12, supporting member 70 may be a ring-shaped member and, more particularly, a plastic ring such as a C-shaped plastic ring having a plurality of holes 94. By using a supporting member 70 including a plurality of holes 94, it is possible to use a medical device (not shown) to allow a suture 88′ to be stitched through the duodenal tissue of the circumferential pouch-cuff 72, into and through each hole 94 of the supporting member 70, and into the tissue of duodenum 16 which lies within and adjacent to the circumference of the supporting member 70, as illustrated in the top view of FIG. 12. After such stitching, the end of the suture 88′ can be knotted to form knots 96 which help to secure the supporting member 70 in place. As illustrated in FIG. 12, such stitching and knotting can be performed around at least a portion of circumference of the supporting member 70 using all or substantially all of the holes 94 of supporting member 70 to secure the circumferential pouch-cuff 72 and supporting member 70 in place. As illustrated in FIGS. 11 and 12, knots 96 can be formed on the inner surface 26 of modified duodenum 16″ and on the outer surface 76 of circumferential pouch-cuff 72 as a result of stitching and knotting.

With further reference to FIGS. 7 to 9, medical device 64 may be any suitable medical device known in the art. A particularly useful medical device 64 may be an endoscopic device. In particular, in some embodiments, to facilitate the engagement of medical device 64 to duodenal tissue, medical device 64 may be an endoscopic device such as an endoscope comprising balloon 66 which can be inflated to engage duodenal tissue about the inner surface 26 of duodenum 16 at a location 85 which is essentially between the papilla of vater 36 and the Ligament Treitz 62, as illustrated in FIG. 7. Desirably, location 85 is several inches distal to the papilla of vater 36 and near the Ligament of Treitz 62, as illustrated in FIG. 7.

Location 85 may be any suitable distance distal to the papilla of vater. Desirably, location 85 is several inches distal to the papilla of vater and near the Ligament of Treitz 62, as illustrated in FIG. 7.

Where medical device 64 comprises an endoscopic device and, more specifically, a balloon scope as illustrated in FIG. 7, the supporting member 70 may be inserted on the outer surface 32 of the duodenum 16 around the circumference of duodenal tissue at the point where the balloon 66 of the balloon scope engages the remaining portion 98 of balloon scope, as illustrated in FIG. 8. With reference to FIG. 8, it will be understood that medical device 64, i.e., the balloon scope including balloon 66 and remaining portion 98 (shown in hatch marks) may be positioned within the lumen 40 of original duodenum 16.

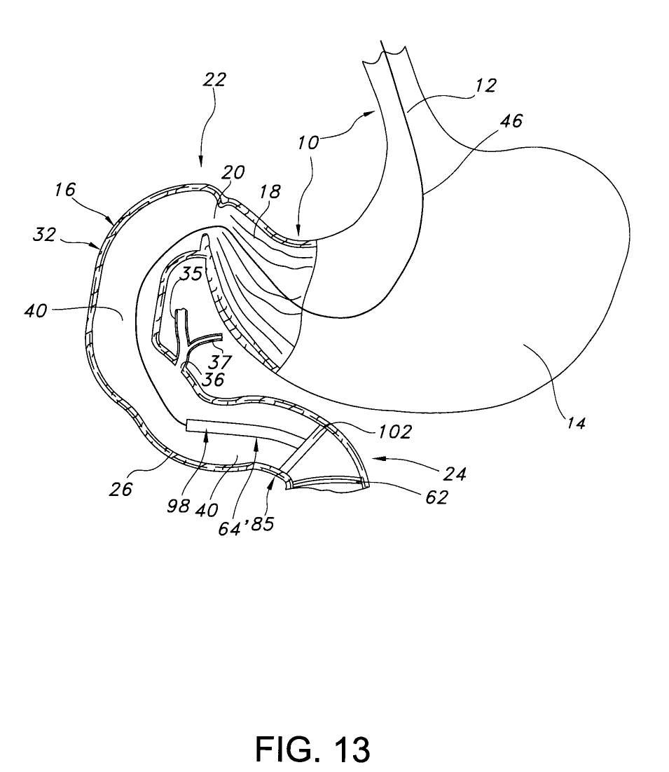

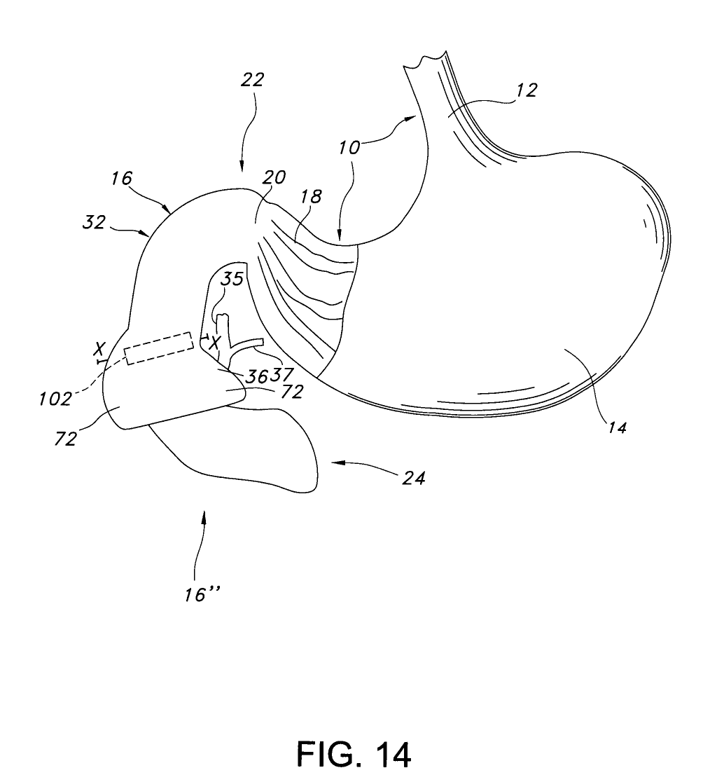

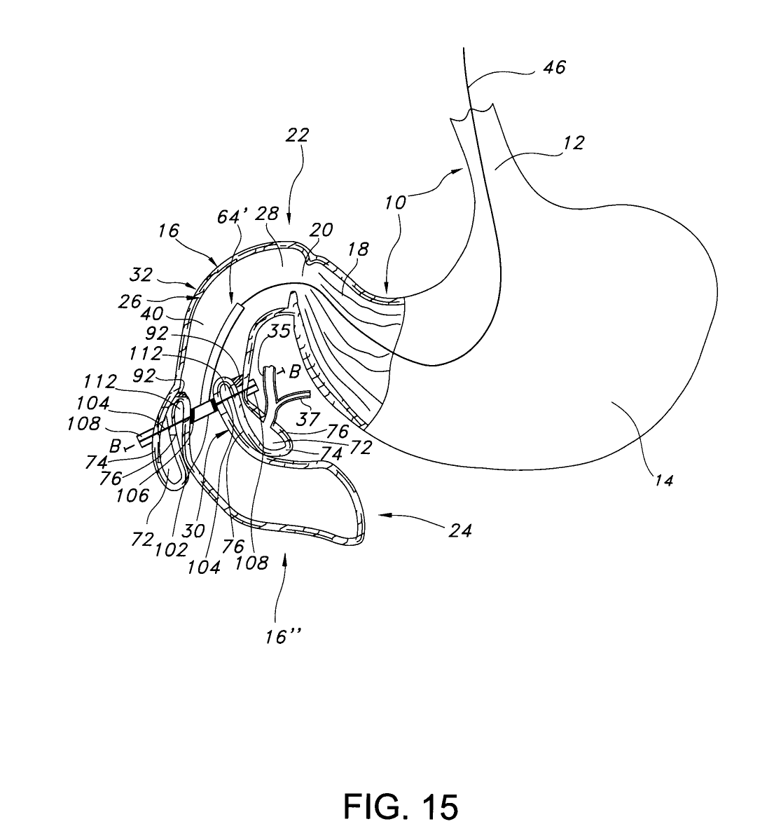

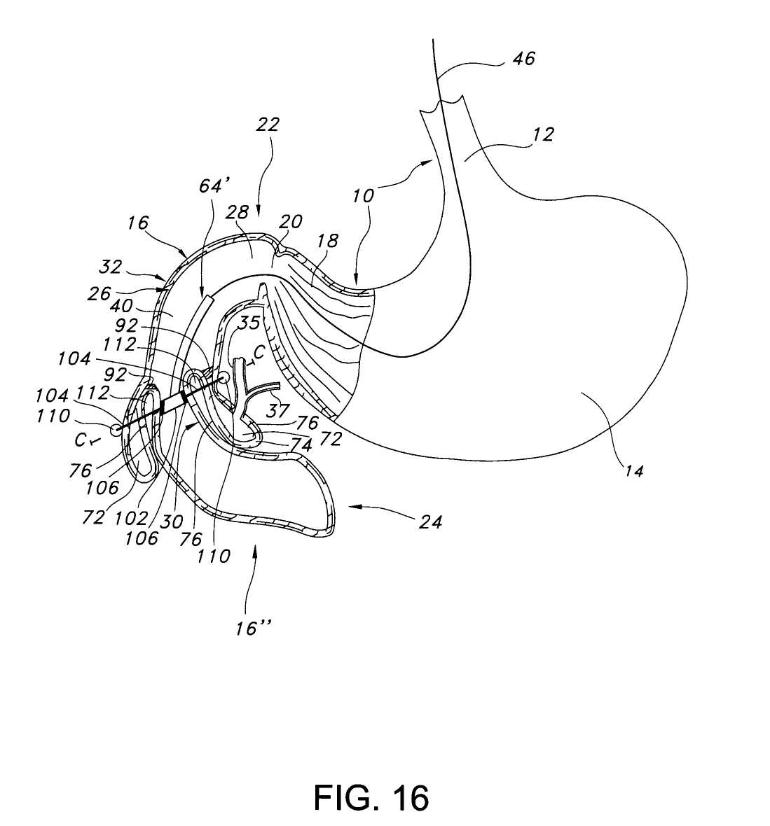

In some embodiments, medical device 64 may comprise a medical device 64′ having a suction device 102 attached thereto to facilitate the engagement of medical device 64′ to the inner surface 26 of duodenum 16 when medical device 64′ is positioned at location 85 essentially between the papilla of vater 36 and Ligament of Treitz 62, as shown in FIG. 13. As illustrated in FIG. 13, location 85 may be near the Ligament of Treitz 62. A partially ring-shaped suction device 102 may facilitate the ability of the medical device 64′ to engage the inner surface 26 of the duodenum 16 about its circumference by means of suction. Other engaging devices such as clamps, forceps or anchors may be used. In accordance with a method of the invention, medical device 64′ may then be retracted using, for example, a guide wire 46, to a location between pylorus 20 and papilla of vater 36, as illustrated in the cross-sectional lengthwise view of modified duodenum 16″ illustrated in FIG. 15. Suction from suction device 102 may assist duodenal tissue from the inner surface 26 of duodenum 16 in being pulled to the aforementioned location, thereby forming a modified duodenum 16″ having at least a partially circumferential pouch-cuff 72, as illustrated in the perspective view of alimentary canal 10 including modified duodenum 16″ illustrated in FIG. 14 and in the cross-sectional lengthwise view of alimentary canal 10 including modified duodenum 16″ illustrated in FIGS. 15 and 16.

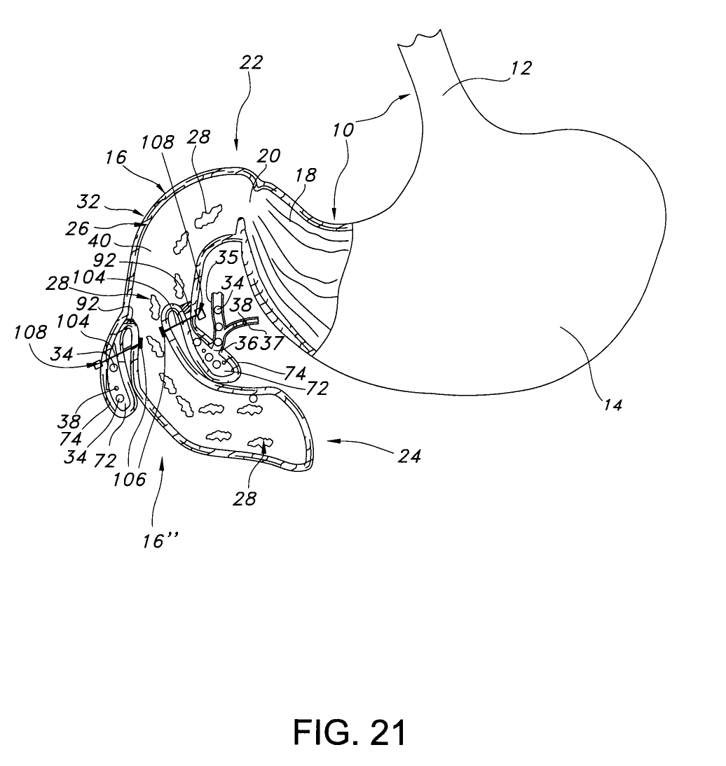

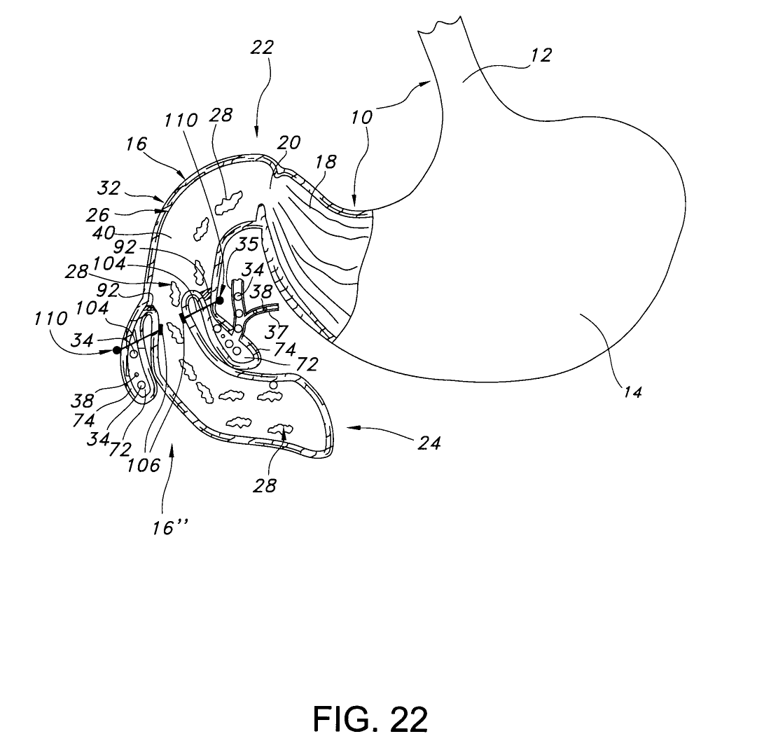

Thereafter, as further illustrated in the cross-sectional FIGS. 15 and 16, an anchor 92, such as an annular anchor, may be positioned on the inner surface 26 of modified duodenum 16″ at a location that is positioned within the inner surface 26 of duodenum 16 at or near the top of the circumferential fold of duodenal tissue which forms the circumferential pouch-cuff 72. Guide wire 46 and medical device 64 can then be removed from alimentary canal 10. Anchor 92 can then be activated such that chyme 28 flowing from the stomach 14 may pass into the original lumen 40 of duodenum 16. Moreover, digestive fluids (i.e., bile fluid 34 and pancreatic fluid 38) may be at least partially prevented from flowing into the original lumen 40 of duodenum 16 and mixing with chyme 28. This is illustrated in FIGS. 21 and 22, respectively, which are lengthwise cross-sectional views of the alimentary canals including modified duodenums 16″ illustrated in FIGS. 15 and 16, respectively, after removal of the ring-shaped suction device 102 and subsequent activation of anchor 92 to prevent chyme from flowing into circumferential pouch-cuff 72. In particular, anchor 92 can be activated such that digestive fluids (i.e., bile 34 and pancreatic fluid 38) flowing through the papilla of vater 36 become trapped in the circumferential pouch-cuff 72 and can be substantially or partially prevented from mixing with chyme 28 which flows from the stomach 14, as illustrated in FIGS. 21 and 22. The digestive fluids (i.e., bile 34 and pancreatic fluid 38) which are trapped in circumferential pouch-cuff 72 are then reabsorbed through the wall 74 of the circumferential pouch-cuff 72.

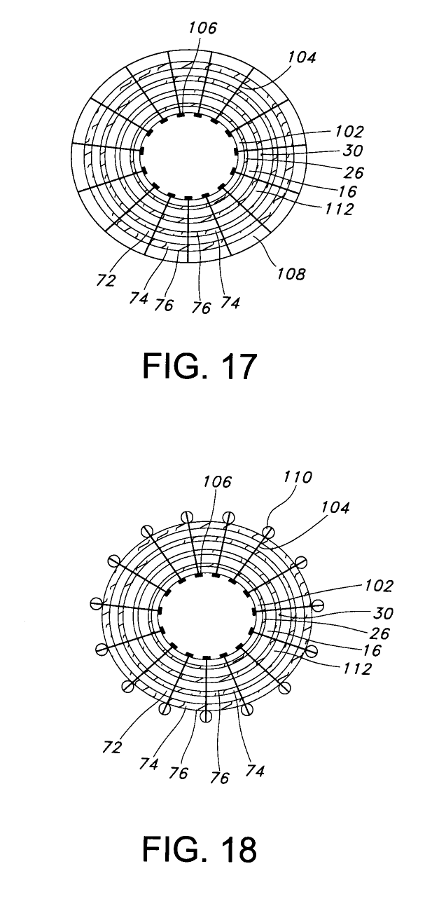

In some embodiments, after ring-shaped suction device 102 is pulled to a location 81 between pylorus 20 and papilla of vater 36 within the original lumen 40 of duodenum 16, ring-shaped suction device 102 may deploy needles 104 around its circumference, as illustrated in FIGS. 17-18, which are exploded top cross-sectional views of modified duodenum 16″ shown in FIG. 14 along line X-X with needles 104 deployed as shown in the lengthwise cross-sectional views of FIGS. 15-16. The needles 104 may puncture the layer 30 of duodenal tissue which does not form a part of the circumferential pouch-cuff 72 and then puncture the wall 74 of the circumferential pouch-cuff 72 about its circumference as shown in FIGS. 15-18. Deploying needles 104 in such a manner through the wall 74 of the circumferential pouch-cuff 72 layer and the underlying duodenal tissue layer 30 may prevent rotation and sliding of those layers relative to each other.

When needles are deployed as described above, in some embodiments, the needles may have a backing 106 as illustrated in FIGS. 15-18 which cannot penetrate the inner surface 26 of the duodenum 16 and which thereby secures the needles 104 to the duodenum 16 and prevents the needles 104 from advancing too far through the circumferential pouch-cuff 72.

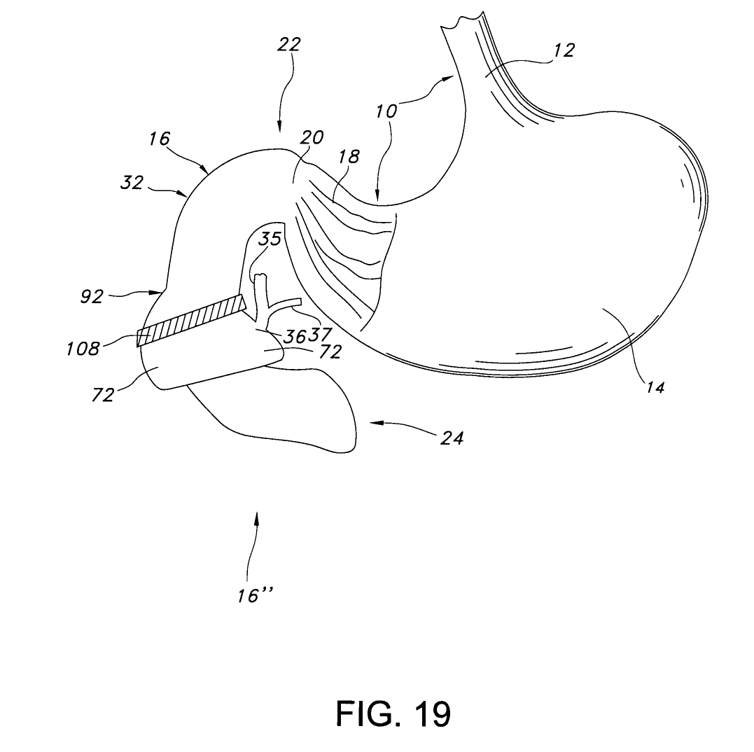

In some embodiments, as illustrated in FIGS. 15 and 17, an open ring 108 which is different from ring-shaped suction device 102 may be inserted on the outer surface 76 of circumferential pouch-cuff 72 such that it attaches to the needles 104 and helps to clasp the duodenal tissue of the circumferential pouch-cuff 72 in place. FIG. 19 illustrates a perspective view of a modified duodenum 16″ having an open ring 108 positioned on the outer surface of circumferential pouch-cuff 72.

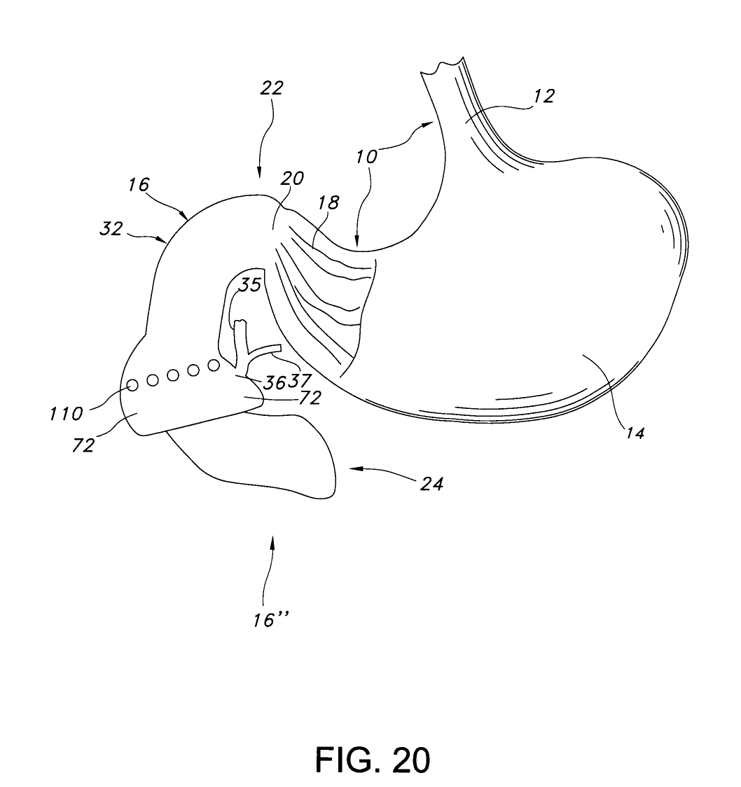

In other embodiments, as illustrated in FIGS. 16 and 18, caps 110 may be placed over the ends of needles 104. FIG. 20 is a perspective view of modified duodenum 16″ having caps 110 placed over the ends of needles 104.

With further reference to FIGS. 15 and 16, it will be understood that needles 104 can be releasably attached to ring-shaped suction device 102. As such, after needles 104 have been deployed and either open-ended ring 108 or caps 110 placed over the ends of needles 104 as described above, needles 104 can be detached from ring-shaped suction device 102, and medical device 64′ with ring-shaped suction device 102 is removed from the inner surface 26 of modified duodenum 16″.

After deployment of needles 104, subsequent removal of the ring-shaped suction device 102, and activation of an anchor 92 as described above, digestive fluids (i.e., bile fluid 34 and pancreatic fluid 38) flowing through the papilla of vater 36 become trapped in the circumferential pouch-cuff 72 and may be significantly prevented from mixing with chyme 28 which flows from the stomach 14, as illustrated in FIGS. 21 and 22.

It will be understood that any suitable fastener may be used to clasp the duodenal tissue of circumferential pouch-cuff 72 in place. In particular, any T-fastener or surgical fasteners may be used to hold the circumferential pouch-cuff 72 in place such as fasteners 138 and 138′ as illustrated in FIGS. 26 and 27, respectively. Suitable T-fasteners 138, 138′ for use in the invention are illustrated in FIGS. 26 and 27, respectively.

With further reference to the circumferential pouch-cuff 72, it will be understood that in some embodiments it may lie directly adjacent to underlying duodenal tissue layer 30. In other embodiments, a space 112 may be present between underlying duodenal tissue layer 30 and the outer surface 76 of the wall 74 of circumferential pouch-cuff 72, as illustrated in the lengthwise cross-sectional views of FIGS. 15 and 16 and in the exploded top cross-sectional views of FIGS. 17 and 18.

With respect to all embodiments of the invention, it will be understood that the connection between tissues may be enhanced with adhesives, energy, cells, biologicals, tissue matrices, and/or combinations thereof. For example, tissue adhesions may be improved by the use of tissue glues, energy (e.g., heat, light) on tissue growth matrices to induce a more secure attachment with the intestinal (such as the duodenal) walls.

The medical device of the invention may be delivered to the duodenum using any suitable delivery device known in the art. In some embodiments, a wire can be used to deliver the medical device to the duodenum. In other embodiments, a rapid exchange catheter such as the rapid exchange catheter disclosed in U.S. Pat. No. 6,592,549, the full contents of which are incorporated by reference herein, may be used. In still other embodiments, delivery of medical device may be through an endoscope. In yet other embodiments, a delivery device is employed which includes a fiber optic or a chip which allows visualization of the placement of the medical device. In still other embodiments, a balloon catheter may be employed to deliver medical device to the duodenum. In still other embodiments, delivery of medical device may be unassisted (i.e., no wire or endoscope is employed). In some embodiments, the procedure may be surgical. In some embodiments, the procedures may be percutaneous or laparoscopic. In yet other embodiments, the procedures may be performed endoscopically or transluminally. In some embodiments, the procedure may employ remotely operated surgical tools such as, for example, robotic tools and direct device endoscopic tools.

Moreover, it will be understood that, in some embodiments, the procedures may be reversible. In particular, in some embodiments, it may be possible to remove common wall 50 and to thereby remove separate channels 42 and 44. Moreover, in some embodiments, it may be possible to remove the circumferential pouch-cuff 72 of the invention. Accordingly, in some embodiments, the partial or substantial separation of digestive fluids (e.g., bile 34 and pancreatic fluid 38) in accordance with the invention may be reversible as may be the slow mixing of digestive fluids (e.g., bile 34 and pancreatic fluid 38).

The invention being thus described, it will now be evident to those skilled in the art that the same may be varied in many ways. Such variations are not to be regarded as a departure from the spirit and scope of the invention and all such modifications are intended to be included within the scope of the following claims. Further, any of the embodiments or aspects of the invention as described in the claims may be used with one and another without limitation.

What is claimed is:

1. A method for inducing weight loss in a patient comprising the steps of:
(i) accessing an intestine having a proximal end, a distal end, an outer surface, and an original lumen extending therethrough; and
(ii) bringing intestinal tissue from different locations on the intestine into abutting relationship along at least a portion of the length of the intestine to form a separate channel within the intestine.

2. The method of claim 1, wherein at least two channels are formed out of said original lumen.

3. The method of claim 2, wherein said at least two channels share a common wall.

4. The method of claim 1, wherein the intestinal tissue from the different locations on the intestine spans a portion of the length of the intestine.

5. The method of claim 3, wherein said common wall comprises a common line of tissue engagement.

6. The method of claim 5, wherein said common line of tissue engagement comprises a tissue-connecting means.

7. The method of claim 6, wherein said tissue-connecting means is selected from the group consisting of sutures, staples, anchors, clips, fasteners, and combinations thereof.

8. The method of claim 3, wherein said common wall separates said original lumen along a portion of the length of said intestine.

9. The method of claim 2, wherein one of said channels receives digestive fluids from the biliary duct and the other of said channels receives chyme from the pylorus.

10. The method of claim 3, wherein said common wall slows the mixing of digestive fluids with chyme.

11. A method for inducing weight loss in a patient comprising the steps of:
(i) grasping an intestine comprising intestinal tissue and having an original lumen across the diameter of said intestine proximate to the distal end of said intestine to bring intestinal tissue from different locations on said intestine into contact; wherein said intestine comprises a papilla of vater and a pylorus;
(ii) employing a tissue-connecting device to attach said intestinal tissue from said different locations on said intestine; and
(iii) continuing to employ said tissue-connecting device to attach intestinal tissue from different locations on said intestine until a location on the outer surface of intestine that is between the papilla of vater and pylorus is reached; wherein steps (i)-(iii) result in the formation of two separated channels within the intestine.

12. The method of claim 11, wherein said at least two channels are formed out of said original lumen.

13. The method of claim 12, wherein said two channels share a common wall.

14. The method of claim 11, wherein the intestinal tissue from the different locations on the intestine spans a portion of the length of the intestine.

15. The method of claim 13, wherein said common wall comprises a common line of tissue engagement.

16. The method of claim 15, wherein said common line of tissue engagement comprises a tissue-connecting means.

17. The method of claim 16, wherein said tissue-connecting means is selected from the group consisting of sutures, staples, anchors, clips, fasteners, and combinations thereof.

18. The method of claim 13, wherein said common wall separates said original lumen along a length of said intestine.

19. The method of claim 12, wherein one of said channels receives digestive fluids from a biliary duct and the other of said channels receives chyme from the pylorus.

20. The method of claim 13, wherein said common wall slows the mixing of digestive fluids with chyme.

21. A method for inducing weight loss in a patient comprising the steps of:
(i) positioning a tissue-connecting device at a location between the pylorus and the papilla of vater on the outer surface of an intestine having an original lumen; wherein said intestine is connected to a papilla of vater and a Ligament of Treitz;
(ii) employing said tissue-connecting device in a radial direction towards the center of said intestine to join tissue from different locations on the intestine together by angling said tissue-connecting device distally relative to the pylorus and then employing said tissue-connecting device along a length of the intestine until a location between the papilla of vater and Ligament of Treitz is reached; and
(iii) continuing to employ said tissue-connecting device along a portion of the length of said intestine until a location near the Ligament of Treitz is reached.

22. The method of claim 21, wherein two channels are formed out of said original lumen.

23. The method of claim 22, wherein said two channels share a common wall.

24. The method of claim 21, wherein the intestinal tissue from the different locations on the intestine spans a portion of the intestine.

25. The method of claim 23, wherein said common wall comprises a common line of tissue engagement.

26. The method of claim 25, wherein said common line of tissue engagement comprises a tissue-connecting means.

27. The method of claim 26, wherein said tissue-connecting means is selected from the group consisting of staples, sutures, fasteners, and combinations thereof.

28. The method of claim 23, wherein said common wall separates said original lumen along a portion of a length of said intestine.

29. The method of claim 22, wherein one of said channels receives digestive fluids from a biliary duct and the other of said channels receives chyme from the pylorus.

30. The method of claim 23, wherein said common wall slows the mixing of digestive fluids with chyme.

31. A method for inducing weight loss in a patient comprising:
(i) accessing an intestine comprising intestinal tissue and having a proximal end, a distal end, and an original lumen extending therethrough, wherein said intestine comprises a pylorus and is connected to a papilla of vater and Ligament of Treitz;
(ii) inserting a medical device into said proximal end of said intestine;
(iii) engaging intestinal tissue at a location that is between the papilla of vater and the Ligament of Treitz by means of said medical device such that said intestinal tissue becomes removably attached to said medical device; and
(iv) retracting said medical device and said tissue attached thereto to a location within said original lumen that is between the papilla of vater and the pylorus; wherein at least a partial circumferential pouch-cuff is formed at said proximal end of said intestine as a result of retracting said endoscopic device.

32. The method of claim 31, wherein the at least partial circumferential pouch-cuff retards chyme from mixing with digestive fluids in the intestine.

33. The method of claim 32, wherein the at least partial circumferential pouch-cuff receives digestive fluids flowing from the papilla of vater.

34. The method of claim 31, further comprising the step of positioning an anchor within said duodenum.

35. The method of claim 31, further comprising positioning a supporting member within said duodenum prior to retracting the medical device.

36. A system for inducing weight loss in a patient comprising:
(a) a grasping means for grasping an intestine and bringing intestinal tissue into abutting relationship; and
(b) a connecting device for attaching in abutting relationship tissues to form a common wall which separates an intestine into at least two channels along a portion of a length of the intestine.

37. The method of claim 6, wherein the tissue-connecting means is temporary or removable.
